(19) United States
(12) Patent Application Publication
(10) Pub. No.: US 2007/0218454 A1
(43) Pub. Date: Sep. 20, 2007

(54) Optical detection cell for micro-fluidics

(76) Inventors: Reid A. Brennen, San Francisco, CA (US); Timothy Beerling, San Francisco, CA (US); Hongfeng Yin, Cupertino, CA (US); Kevin P. Killeen, Woodside, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/378,760

(22) Filed: Mar. 16, 2006

(51) Int. Cl. C12Q 1/00

(52) U.S. Cl. 435/4

(57) ABSTRACT

The present invention relates to an optical detection cell for micro-fluidics. The detection cell provides a first layer, a detection cell layer contacting the first layer, a third layer contacting the detection cell layer and a detection channel defined through the detection cell to serve as a light path for receiving light for detecting a molecule Methods of detecting molecules and making the detection cell are also disclosed

### BACKGROUND

Various detection devices and detection cells have been designed for identifying and characterizing small molecules. Typical devices may include, UV Vis, fluorimeters or micro-fluidic devices. Most of these devices provide some type of detection cell with limited volume for holding the sample while light is passed through the cell. This allows for conservation of sample and increase of signal to noise (i.e. improve characterization and detection).

Most of these devices and cells operate by first placing a buffer or a fluid medium in the detection cell. Then light of a defined wavelength is passed through the medium and the properties recorded. Next, a sample is then typically dissolved in the same fluid medium and the combined mixture is placed in the detection for a reading. Various light wavelengths can then be passed through or scanned through the device.

More recently, micro-fluidic devices are being used in identifying and characterizing small molecules. These devices avoid the problem of having to use large amounts of sample, transfer sample and take multiple readings to remove baseline contamination readings or low signal to noise. Smaller and smaller samples have been detected, characterized and recaptured using these devices.

Many of the mentioned ultraviolet and visible absorption methods adhere to the Beer Lambert law. The Beer Lambert Law provides that:
ε×b×C=A (1)
where C is the concentration in moles per liter and is assumed to be constant, A is the minimum detectable absorbance, ε is the molar extinction coefficient and b is the path length (typically 1.0 cm). As one will note from this law that as the concentration C or the path length b are increased the absorbance also increases. In other words the minimum level of detection is increased.

With micro-fluidic devices there are additional parameters that must be considered. For instance, path length (L), the volume (V) as well as well as the cross-sectional area (CSA) of the detection cell are also important in effecting the sensitivity level. Ideal conditions for improving the signal to noise ratio (sensitivity) require decreasing V, increasing L and decreasing CSA. This provides the optimal conditions for obtaining the best sensitivity. However, many detection cells or devices do not allow for improving each of these parameters. Typically the improvement of one condition causes a negative effect on the other parameters. In the end this does not improve overall sensitivity levels. For this reason there is a need to improve the overall signal to noise ratios of detection devices and detection cells. In addition, it would be desirable to provide a detection device or cell that minimizes overall sample volume, yet increases L and decreases CSA. To date few devices and/or detection cells provide the ability to improve each of these parameters to provide improved sensitivity. In addition, most of the present detection devices and detection cells do not provide flexibility for improving these parameters. Of the limited designs that do, most are also fairly cost prohibitive. These and other problems experience by the prior art have been obviated by the present invention.

### SUMMARY OF THE INVENTION

The present invention relates to an apparatus and method for detecting a molecule. The detection cell of the present invention provides a first layer or substrate, a detection layer or substrate contacting the first layer; a third layer or substrate contacting the detection cell layer; and a detection channel defined through the detection cell to serve as a light path for receiving light for detecting a molecule.

The invention also provides a method for detecting a molecule. The method comprises transmitting light at a molecule in a detection channel and detecting the molecule in the detection channel.

### BRIEF DESCRIPTION OF THE FIGURES

The invention is described in detail below with reference to the following figures:

FIG. 1 shows a general perspective view of an embodiment of the present invention.

FIG. 2 shows a cross sectional view of a first embodiment of the present invention.

FIG. 3 shows a cross-sectional view of a second embodiment of the present invention.

FIG. 4 shows a cross-sectional view of a third embodiment of the present invention.

FIG. 5 shows a cross-sectional view of a fourth embodiment of the present invention.

### DETAILED DESCRIPTION OF THE INVENTION

Before describing the invention in detail, it must be noted that, as used in this specification and the appended claims, the singular forms “a,” “an,” and “the” include plural referents unless the context clearly dictates otherwise. Thus, for example, reference to “a layer” includes more than one “layer”, reference to “a substrate” includes more than one “substrate”.

In describing and claiming the present invention, the following terminology will be used in accordance with the definitions set out below.

The term “reconfigurable” refers to a substrate or layer that is capable of being assembled or reconfigured into a larger structure. A detection layer may comprise various substrates or layers that may be assembled to define the detection layer. The parts may be similar in design or different. In certain embodiments the substrates may comprise similar monolithic designs that may provide for predictable reconfiguration of the detection cell. For instance, a certain molecule may be first tested using a defined number or substrates. The detection cell may then be reconfigured in a quick and efficient manner by adding or removing additional substrates. A different molecule can then be tested at a different path length defined by the number of substrates. In certain cases the same molecule may also be tested at different path lengths defined by reconfigured detections cells to find the best path length and configuration for detecting and characterizing molecules.

The term “detection cell” refers to an enclosed or partially enclosed area capable of being used to hold and analyze a sample. Typical detection cells may comprise one or more layers or substrates with one or more detection channels that allow for the transmission of light to the sample.

The term “detection device” refers to a device that may comprise one or more detection cells.

The term “detection layer” refers to a uniform or non-uniform material that may comprise a substrate or a portion of a substrate.

The term “detection channel” refers to an area, chamber, or elongated space or conduit capable of holding and/or allowing for sample movement and/or detection. Detection channel(s) typically are designed within a detection cell or detection device. It is within the scope of the invention to provide multiple detection channels within a single detection cell or detection device.

The term “fluidic communication” refers to allowing fluid to pass between structures. Samples and/or liquid can also be moved from place to place.

The term “light” refers to matter that has both wave and particle properties. Typical light used may include and not be limited to ultraviolet light, visible light, infrared, fluorescence light, and bioluminescence light.

The term “light path” refers to the path along which light may travel for detecting a molecule. This may include transmission or reflection.

The term “micro-fluidic” refers to devices that are small in scale.

The term “molecule” refers to any material capable of being detected by light transmission, absorbance or reflection.

The term “monolithic” refers to a single structure comprising a homogenous material.

The term “opaque material” refers to a material that prevents or allows only limited light transmission.

The term “passes through” refers to a channel or channel portion that may continue from one side to the other side of a layer or substrate or may intersect a portion of a layer or substrate.

The term “substrate” refers to a structure capable of comprising a uniform material or one or more layers of material.

The term “transparent material” refers to a material capable of allowing light to pass through it.

The invention is described herein with reference to the figures. The figures are not to scale, and in particular, certain dimensions may be exaggerated for clarity of presentation.

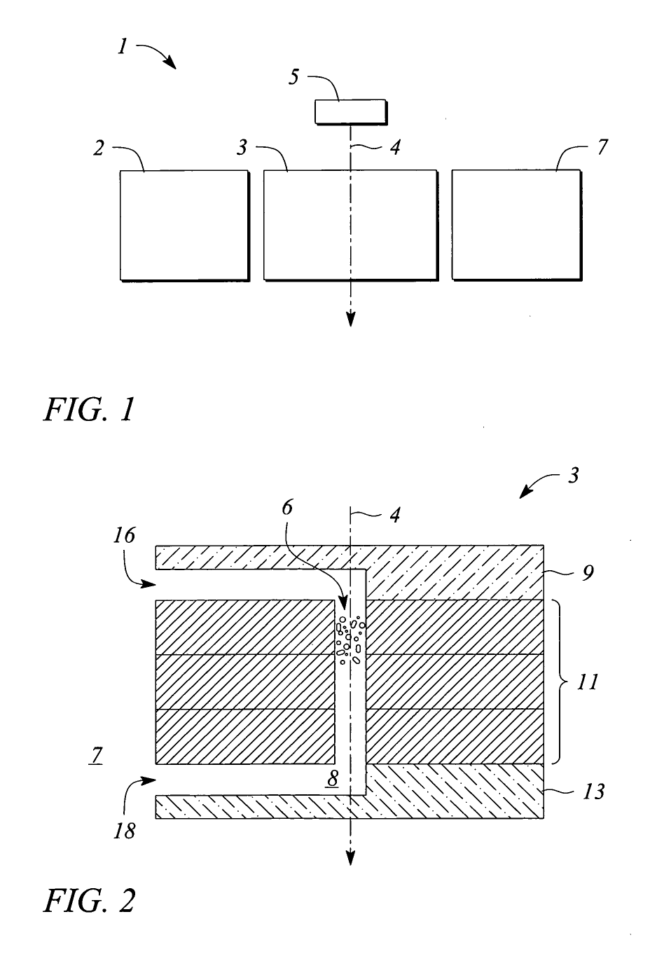

FIG. 1 shows a diagram of a general perspective view of the detection device 1 of the present invention. The detection device 1 comprises an input device 2, an optical detection cell 3, a light source 5 and a detector 7. The detector 7 is generally positioned adjacent to the detection cell 3. Although the figures show a single detection cell 3 multiple detection cells 3 may be employed with the present invention. These detection cells 3 may be employed in various arrangements and orientations. They may also be spaced in parallel or in series. It is within the scope of the invention to use multiple multiple light sources 5, multiple input devices 2 and multiple detectors 7 in various arrangements and configurations. For instance they may be positioned also in series or parallel orientations. Also within the scope of the invention is to employee multiple detection devices 1 in various configurations. This would include and not be limited to orienting various detection device 1 in tandem, parallel and/or series. Other configurations not discussed or disclosed are also within the scope of the invention.

The input device 2 may comprise any device used for holding or transporting a sample to a micro-fluidic device or similar type device. Input devices are well known in the art. These devices may comprise and are not limited to capillaries, micro-fluidic devices and micro-fluidic chips. It should be noted that input devices include and are not limited to micro-sized devices.

The light source 5 may comprise any number of light sources known in the art that may be used to identify or characterize a molecule. Light sources are well known in the art which light may be reflected, scattered absorbed, or absorbed and re-emitted (fluorescence) by the various molecules. In particular, light sources may include and not be limited to sources that provide infrared, visible, ultraviolet or other particular wavelengths of light.

The detector 7 may comprise any number of common or well known detectors in the art that may be used for detecting light that has been reflected, transmitted, absorbed or scattered from small molecules placed in the detection device 1.

Detector 7 should not be confused with second detector 12. The second detector 12 may comprise any number of analytical or instrumental device or devices used for further characterizing, isolating or separating a molecule. For instance, in certain instances it could be imagined that the detector 12 comprises a mass spectrometer or mass spectrometry system, a microarray device, a gel, an isoelectric focusing device, a separation device, a 2-dimensional or 3-dimensional gel, a flame or furnace atomic absorption device, an NMR or EPR device, an HPLC column or device or any other device or combination of the above devices that may further help characterize, identify or capture the molecules 6.

FIG. 2 shows a cross-sectional view of a first embodiment of the invention. The detection cell 3 comprises a first layer 9, a detection layer 11, and an optional third layer 13. The detection cell 3 provides a detection channel 8 that is designed for receiving a molecule 6. The detection channel 8 defines the light path or a portion of the light path 4 that the molecule 6 may be identified and/or detected. The size of the light path 4 can be defined or determined by the number of layers or substrates employed in the detection layer 11. The detection layer 11 is important to the invention. The detection layer 11 allows for the flexible construction of the detection cell 3. For instance, 1-15 layers or substrates may be employed to build the detection layer 11. By varying the number of layers or substrates it is possible to change and define the length of the detection channel 8 and/or the light path 4. Being able to alter or define the detection channel 8 and light path 4 is important to the invention. This design provides the ability to increase the overall path length of the light being transmitted through the detection cell 3 to detect the molecule 6. It is particularly important to be able to increase the overall light path length (L) while at the same time reducing the volume (V) of the detection channel 8. In addition, the cross sectional area of the channel (CSA) may be reduced. As a result, the overall sensitivity or signal to noise ratio is improved. It should also be noted that molecules 6 may be in static or dynamic movement after entering the detection channel 8.

The first layer 9 may comprise any number of materials that are transparent to light in the desired spectrum. For instance, the first layer 9 may comprise a material selected from the group consisting of silicon dioxide, sapphire, Pyrex™, a transparent polymer, a silica wafer or a quartz material. Other materials known in the art may be employed. Also other materials not described here may be employed. An important functional aspect of the first layer 9 is its ability to allow light or a portion of light to pass through it. Typically the first layer 9 comprises a transparent material. In certain instances, the first layer 9 may comprise a portion of a substrate or the whole substrate. The first layer 9 may comprise other materials or may be monolithic in design. The first layer 9 may also comprise a top surface 20.

The detection cell layer 11 contacts the first layer 9. The detection layer 11 may comprise from 1-15 layers or substrates. The thickness of the detection cell layer 11 can therefore range from about 0.1 to 10 millimeters depending upon the number of layers and/or substrates employed. Ideally, the detection layer 11 may comprise from 1-5 layers or substrates. Each layer may vary in size or be consistent in thickness throughout the entire detection cell layer 11. In addition, the detection cell layer 11, may comprise a single material or multiple materials. The composition may be composite, homogenous or heterogenous. The substrate may be monolithic or fragmented into various sections or sub-sections. The detection layer 11 may comprise a portion of a substrate. The detection cell layer 11 may also comprise a transparent or opaque material. The detection cell layer 11 may comprise various semiconductor materials that make construction and assembly of the detection cell easy. The detection cell layer 11 may comprise a material selected from the group consisting of a metal, a glass, a ceramic, or a polymer, or a combination of the above. Examples included silicon, silicon dioxide, silicon carbide, nickel, tungsten, Pyrex™, sapphire, sintered or monolithic ceramic, polyimide, PEEK (polyetheretherketone), and the like. For instance, the detection cell layers may be designed in various ways so that they may be assembled or disassembled quickly and at defined thickness. This allows for designing and testing using various light sources and path lengths within the same detection cell 3. It also provides for added flexibility to maximize the detection of a particular molecule without having to redesign the whole detection cell 3.

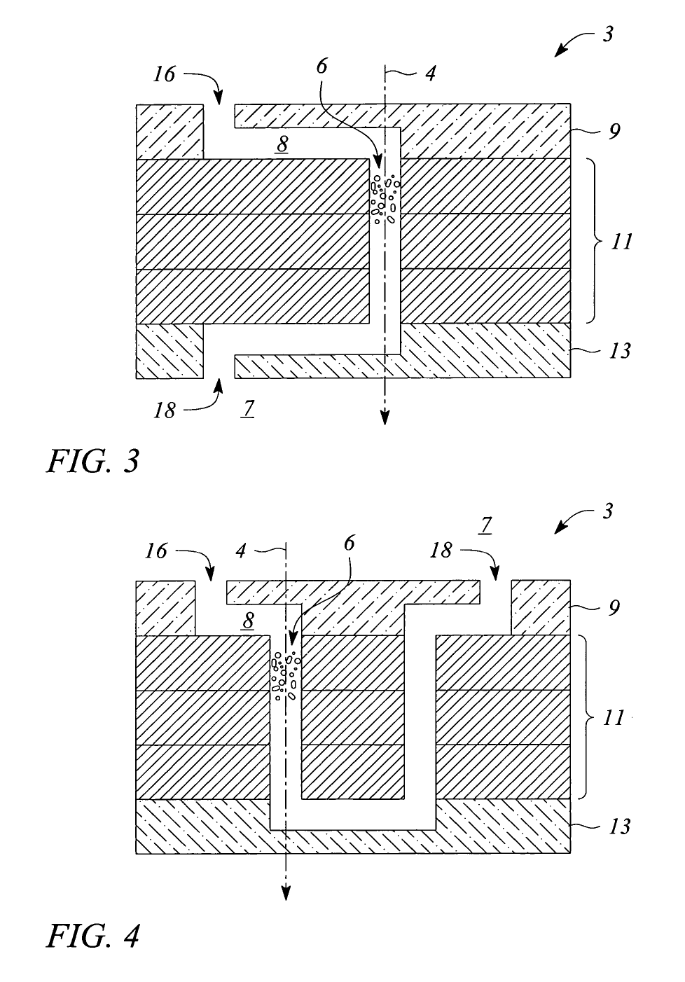

The third layer 13 is optional to the present invention. In certain instances and embodiments it may contact the detection layer 11. However, this is not a requirement of the invention. It other embodiments the third layer 13 may be eliminated. The third layer 13 may comprise various layers or substrates. The actual width or thickness of the material may be adjusted. The third layer 13 may comprise a number of materials that are transparent to light. For instance, the third layer 13 may comprise a material selected from the group consisting of a metal, a glass, a ceramic, or a polymer, or a combination of the above. Examples include silicon, silicon dioxide, silicon carbide, nickel, tungsten, Pyrex™, sapphire, sintered or monolithic ceramic, polyimide, PEEK (polyetheretherketone), and the like. Other materials known in the art may be employed. Also other materials not described here may be employed. An important functional aspect of the third layer 13 is its ability to allow light or a portion of light to pass through it. Typically the third layer 13 comprises a transparent material. In certain instances, the third layer 13 may comprise a portion of a substrate or the whole substrate. FIG. 2-4 show the third layer 13 comprising a portion of a larger substrate. The third layer 13 may comprise other materials or may be monolithic in design. In some embodiments the third layer 13 may comprise a bottom surface 22.

The detection channel 8 is defined by the layers and/or substrates comprising the detection cell layer 11. The term detection channel 8 may be interpreted to mean a channel having any number of lengths, widths or volumes. The detection channel 8 may also be defined by the first layer 9 and the third layer 13. However, this is not a requirement of the invention. In certain embodiments one or more detection channels 8 may be employed within the present invention. The detection channel 8 may have an inlet port 16 and an exit port 18 (See FIGS. 2-4). The inlet port 16 and exit port 18 may pass through any number of layers including and not limited to the first layer or substrate, the detection layer or substrate and the third layer or substrate. Multiple ports may be employed and they may be positioned on the same layer or substrate or different layers or substrates. They way vary in size and dimension.

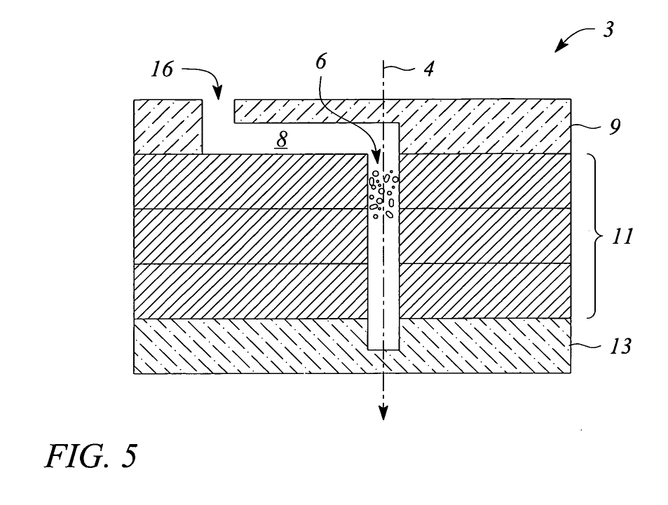

FIG. 5 shows an embodiment where the detection channel only has an inlet port 16. The light used in the detection is generally transmitted down the light path 4 where it reflects off of a substrate or layer and is transmitted or reflected back to a detector or other device for identifying or characterizing a molecule 6. The internal volume, shape and length of the light path 4 and detection channel 8 can vary. This is an important aspect of the invention. The detection channel 8 may contain a volume of from about 10 to 1000 nanoliters of fluid. Typical flow rates through the channel may vary but can range from around 40-4000 nanoliters/minute.

FIGS. 2-4 show various embodiments of the invention with an altered detection channel 8. Other embodiments and designs may be employed with the present invention.

Having described in detail the apparatus of the invention, a brief description of the method is now in order.

Methods of detecting molecules and making the detection cell will now be described. The method of detecting a molecule 6 is accomplished in a simple manner. Referring now to FIGS. 1-4, the molecule 6 is first input at the inlet port 16 of the optical detection cell 3. The molecule 6 then travels down the detection channel 8. This may be accomplished in a number of ways. Fluids or other mediums may be employed. In addition, if a fluid or other medium is employed it may be in a static or dynamic state. After the molecule 6 has entered the detection channel 8 it is transported to an area that is accessible to light. First layer 9 comprises a transparent material that allows for light to pass from a light source 5 into the detection channel along the light path 4. Typically, light path 4 is created in a portion of the detection channel 8. The light can then impinge on a molecule 6 or be used to detect a molecule 6 that is positioned in the detection channel 8 along the light path 4. This is generally accomplished by measuring the light that is absorbed by the molecule 6 while passing through the optical detection cell 3 or by detecting light emitted or re-emitted by the molecule 6. A method of detecting the molecule 6 comprises transmitting light to a molecule 6 in a detection channel 8 of a detection cell 1, and detecting the molecule in the detection channel 8. The method of making the detection cell 3 comprises a simple process. The method comprises providing a first layer 9, providing a detection cell layer 11, contacting or bonded to the first layer 9, providing a third layer 13 which contacts or is bonded to the detection cell layer 11 and defining a detection channel 8 through the optical detection cell 3 to serve as a light path 4 for receiving light for detecting a molecule 6.

We claim:

1. A detection cell for receiving and detecting a molecule, comprising:
(a) a first layer;
(b) a detection cell layer contacting the first layer;
(c) a third layer contacting the detection cell layer; and
(d) a detection channel defined through said detection cell to serve as a light path for receiving light for detecting a molecule.

2. A detection cell as recited in claim 1, wherein said first layer comprises a transparent material.

3. A detection cell as recited in claim 1, wherein said first layer comprises a portion of a substrate.

4. A detection cell as recited in claim 1, wherein the first layer comprises a monolithic substrate.

5. A detection cell as recited in claim 1, wherein said first layer comprises a material selected from the group consisting of silicon dioxide, sapphire, glass, polymer, or quartz.

6. A detection cell as recited in claim 1, wherein the detection cell layer comprises a portion of a substrate.

7. A detection cell as recited in claim 1, wherein the detection cell layer comprises a monolithic substrate.

8. A detection cell as recited in claim 7, wherein the detection cell layer comprises an opaque material.

9. A detection cell as recited in claim 7, wherein the detection cell layer comprises a transparent material.

10. A detection cell as recited in claim 1, wherein the detection cell layer comprises a monolithic substrate.

11. A detection cell as recited in claim 1, wherein the detection cell layer comprises a semiconductor material.

12. A detection cell as recited in claim 1, wherein the detection cell layer comprises a second layer.

13. A detection cell as recited in claim 1, wherein the detection cell layer comprises from 1 to 15 layers.

14. A detection cell as recited in claim 1, wherein the detection cell layer comprises from 1 to 15 substrates.

15. A detection cell as recited in claim 1, wherein the third layer comprises a portion of a substrate.

16. A detection cell as recited in claim 1, wherein the third layer comprises a monolithic substrate.

17. A detection cell as recited in claim 1, wherein the third layer comprises an opaque material.

18. A detection cell as recited in claim 1, wherein the third layer comprises a transparent material.

19. A detection cell as recited in claim 1, wherein the third layer comprises a material selected from the group consisting of silicon dioxide, sapphire, a transparent polymer, or a quartz material.

20. A detection cell as recited in claim 1, wherein a portion of the detection channel passes through the first layer.

21. A detection cell as recited in claim 20, wherein the detection channel passes through the top surface of the first layer.

22. A detection cell as recited in claim 1, wherein a portion of the detection channel passes through the detection cell layer.

23. A detection cell as recited in claim 1, wherein a portion of the detection channel passes through the third layer.

24. A detection cell as recited in claim 22, wherein the detection channel passes through the bottom surface of the third layer.

25. A detection cell as recited in claim 1, wherein said light for detecting said molecule comprises ultraviolet light.

26. A detection cell as recited in claim 1, wherein said light for detecting said molecule comprises infrared light.

27. A detection cell as recited in claim 1, wherein said light for detecting said molecule comprises visible light.

28. A detection cell as recited in claim 1, further comprising a second detection channel defined through said detection cell to serve as a light path for receiving light for detecting a molecule.

29. A detection cell as recited in claim 1, wherein the molecule being detected comprises a biomolecule.

30. A detection cell as recited in claim 1, wherein the detection channel comprises a volume of from 5 to 1000 nanoliters.

31. A detection cell as recited in claim 1, wherein the detection cell layer is from 0.1 to 10 millimeters in thickness.

32. A detection cell as recited in claim 1, wherein the light path is from 0.1 to 10 millimeters in thickness.

33. A detection device for detecting a molecule comprising:
(a) an input device,
(b) a first detection cell downstream from the input device for detecting a molecule; and
(c) a second detection cell downstream from the first detection cell for detecting a molecule.

34. A detection device for detecting a molecule, comprising:
(a) an input device,
(b) a first detection cell disposed in the detection device; and
(c) a second detection cell disposed in parallel to the first detection cell.

35. A method of making a detection cell for detecting a molecule comprising:
(a) providing a first layer;
(b) providing a detection cell layer contacting the first layer;
(c) providing a third layer contacting the detection cell layer; and
(d) defining a detection channel through the detection cell to serve as a light path for receiving light for detecting a molecule.
